(19) United States
(12) Patent Application Publication
(10) Pub. No.: US 2025/0188397 A1
(43) Pub. Date: Jun. 12, 2025

(54) A BIOREACTOR SYSTEM

(71) Applicant: ORIBIOTECH LTD, London, Greater London (GB)

(72) Inventor: Farlan Veraitch, Greater London, London (GB)

(21) Appl. No.: 18/840,795

(22) PCT Filed: Feb. 21, 2023
(86) PCT No.: PCT/GB2023/050387

§ 371 Date: Aug. 22, 2024

(30) Foreign Application Priority Data
Feb. 22, 2022 (GB) 2202344.4

(51) Int. Cl. C12M 3/00; C12M 1/00; C12M 1/24

(52) U.S. Cl. CPC C12M 23/44; C12M 23/08; C12M 23/14; C12M 23/26

(57) ABSTRACT

A bioreactor system is disclosed, comprising a plurality of pods. The pods are suitable for the culture and maintenance of biological samples. Each pod comprises a housing and a biological sample vessel removably disposed within the housing. The biological sample vessel comprises an interfacing element to provide fluid communication to the biological sample vessel. The plurality of pods are arranged in an array in the bioreactor system. A method of culturing a biological using the bioreactor system, and a kit of parts to form the bioreactor system is also disclosed.

### CROSS-REFERENCE TO RELATED APPLICATIONS

This application is a national phase entry under 35 U.S.C. § 371 of International Patent Application PCT/GB2023/050387, filed Feb. 21, 2023, designating the United States of America and published as International Patent Publication WO 2023/161615 A1 on Aug. 31, 2023, which claims the benefit under Article 8 of the Patent Cooperation Treaty of U.K. Patent Application Serial No. 2202344.4, filed Feb. 22, 2022.

### TECHNICAL FIELD

This disclosure relates to a bioreactor system comprising a plurality of pods in an array.

### BACKGROUND

As cell and gene therapy, as well as other advanced biological therapies, become accepted and available, the challenge of scale-up and streamlining of manufacture increases. Often these treatments are tailored and bespoke for an individual, therefore simply increasing the volume of manufacturing vessels does not solve the problem. Instead, it is necessary to operate multiple individual systems, in which a tailored therapy for each individual is cultured.

This creates a new problem of increased cost and complexity in running multiple independent bioreactor systems. These systems also occupy a large footprint therefore increasing costs in the size of facility required to increase throughput. Likewise, additional employees are required, each having achieved a significant competence level, in order to service, maintain, and operate each of the independent bioreactors.

### BRIEF SUMMARY

In accordance with the present disclosure, there is provided a bioreactor system, comprising: a plurality of pods, the pods suitable for the culture and maintenance of biological samples, each pod comprising: a housing, a biological sample vessel removably disposed within the housing, the biological sample vessel comprising an interfacing element to provide fluid communication to the biological sample vessel, wherein the plurality of pods are arranged in an array. Unitizing each bioreactor as a pod allows for multiple bioreactors to be serviced and housed via the array. The array allows stacking of the bioreactors into a single block, thereby minimizing foot-print of the system and easily allowing scale up. The array can be increased or decreased in size according to manufacturing need. The array can, by way of non-limiting example, comprise 2 or more pods, 3 or more pods, 4 or more pods, 5 or more pods, 10 or more pods, 20 or more pods.

Preferably, the interfacing element provides fluid communication to one or more of: an auxiliary vessel, a gas regulatory system, a waste management system, a sterilization system, and a reservoir containing culture fluid. The interfacing element in each pod connects the interior of the biological sample vessel to services in the outside world, through the pod and to one or more service points in the array. In this way additives, consumables, or essential supplies can be added/removed from each vessel in order to allow the maintenance of the biological sample under culture in the pod located in the array.

Suitably the biological sample vessel is a flexible container thereby allowing for changes in volume within the vessel and avoiding changes in pressure due to fluids being added/removed. Flexible vessels can also accommodate varying sizes of biological samples, whether that is by accommodating samples of initially different physical size or to accommodate a change in biological sample size, such as by expanding the number of cells in culture within a vessel (i.e., physical change in the size of a given biological sample).

Advantageously, each flexible container comprises a biological sample. Containing the biological sample within the flexible container allows for easy location of the biological sample within a pod, and subsequent removal.

Optionally, each flexible container is removably attached to each pod. Accordingly, biological samples may be removed and replaced in a pod, to allow quality testing of a biological sample, for example.

Preferably, the interfacing element comprises an interface plate, coupled to the flexible container, including at least one port. Preferably, the interface plate is the same in each pod located in an array. Standardizing the interfacing element in this way allows any given pod located in an array to receive any biological sample vessel provided that sample vessel is compatible with the interfacing element. The interfacing element is therefore a common interfacing element. A plurality of ports is advantageous in allowing different fluids to be added or removed from the biological sample vessel. Furthermore, where the pods are removable from the array, a common pod interface plate may be provided allowing location of a given pod at any location in an array sharing a complementary pod interface plate.

Advantageously, each location of each of the pods in the array is uniquely addressable thereby allowing an automated system to located and identify a given pod in the array. This also provides the option for applying different culture regimes (e.g., varying amounts and kinds of fluids) to any of the pods located in a single array.

Preferably, each of the plurality of pods is removably located within the array allowing pods to be removed for maintenance without having the disconnect the whole array or moved between arrays in remotely located facilities. Suitably, the pods are removably attached within the array, and optionally wherein the pods may be removably attached from either a first side of the array or from a second side of the array, the first and second sides opposite one another. Advantageously, the array may comprise two sides opposite one another, and the pods may be removable from either side of the array (i.e., from either the first side of the array or the second side of the array). If the first face of the array faces into a clean room and a second face of the array faces into a service area, then a pod may be readily removed into either the clean room or the service area. The array may be further equipped with suitable seals to allow this removable or replacement of pods from either face of the array without compromising a sterile environment inside the clean room. Any suitable means may be employed to locate the pod in the array or to maneuver the pod to either the first or second sides of the array, such as, but not limited to rails or the like.

Suitably, an interior of each of the plurality of pods is isolated from the interior of the other pods in the array. While each pod may receive services from a common source in the array the source may be split such that no communication between the interior of each pod is permitted ensuring there is no risk of cross contamination and also facilitating independent supply of services to each pod allowing for tailored, independent, culture conditions to be created within each pod when necessary.

Preferably, the array is formed integrally with a building, or alternatively the bioreactor system is provided in a mobile facility. As such the array may be provided as part of a static, purpose-built facility or factory ensuring long term stability for biological sample culture. In the case of a mobile facility, the bioreactor system may be deployed at will to culture biological samples in any suitable location, thereby providing cultured products geographically close to the point of use. Such mobile systems may facilitate maintenance and increase system redundancy (due to there being multiple mobile systems with systems in reserve) thereby ensuring a stable supply of cultured samples.

Suitably, the plurality of pods arranged in the array form rows and/or columns. Alternatively, the array may be formed of a less regular arrangement. For the avoidance of doubt it is possible for an array to comprise a row(s) and not a column(s) (excluding a column 1 pod high) or vice versa. Likewise, it is possible for an array that is less regular (“irregular”) to comprise a row(s) and/or a column(s) either totally or in part. Optionally, the array is any of a 2D array, a 3D array, a horse-shoe array, a spherical array, an irregular array. The array may take on any suitable space to effectively fill the volume of the space in which the array is situated. It will be apparent that any suitable arrangement may be used to locate the pods and form an array. Here a 2D array is one in which the plurality of pods are arranged in the same plane and a 3D array is one in which each of the pods are arranged with variable x, y, and z positions if their positions were described with respect to axes, compared to the 2D array where the z position would be fixed for all the pods. A horse-shoe array is where a, in a cross-sectional view, the array substantially conforms to the shape of a horseshoe (i.e., “U” shaped). A spherical array is where the arrangement of the pods corresponds to approximate a sphere. Arrays where the pods are arranged to approximate other 3D shapes are also envisioned, such as a cylinder, circle, and other prisms or geometric shapes.

A method of culturing a biological sample using the bioreactor system is also disclosed. The method comprises loading at least one biological sample into at least one pod, arranging the at least one pod in an array, and culturing the at least one biological sample. The order of the method is not fixed and may be carried out in any logical order, such as arranging the pod in the array and then introducing the sample after, for example.

Advantageously, the method may further comprise one or more of: loading at least one consumable product into one or more pods; removing at least one consumable product from one or more pods; retrieving at least one consumable product from storage; removing at least one consumable product to waste; loading at least one auxiliary service into a pod; retrieving a test sample from the biological sample vessel; and loading a sensor or test apparatus to perform an observation on a condition inside a pod. In providing these extra ancillary services, the up-keep and maintenance of an array may be facilitated thereby reducing time spend by a user as well as minimizing the risk of user-error.

Preferably, the consumable product is either a container for fluid dispensation or a container for sample collection in order to allow culture media to be exchanged within the biological sample vessel, for example, or to allow extraction of an aliquot for testing and monitoring of the culture and conditions inside the biological sample vessel.

A kit of parts to form a bioreactor system is also disclosed in this disclosure. The kit comprises a plurality of pods, the pods suitable for the culture and maintenance of biological samples, each pod comprising: a housing, a biological sample vessel removably disposed within the housing, the biological sample vessel comprising an interfacing element to provide fluid communication to the biological sample vessel.

### BRIEF DESCRIPTION OF THE DRAWINGS

An embodiment of the disclosure is further described hereinafter, by way of example, with reference to the accompanying drawings, in which:

FIG. 1 is a perspective view of a pod;

FIG. 2 is perspective views of an array; and

FIG. 3 is a perspective view of a pod servicing system.

### DETAILED DESCRIPTION

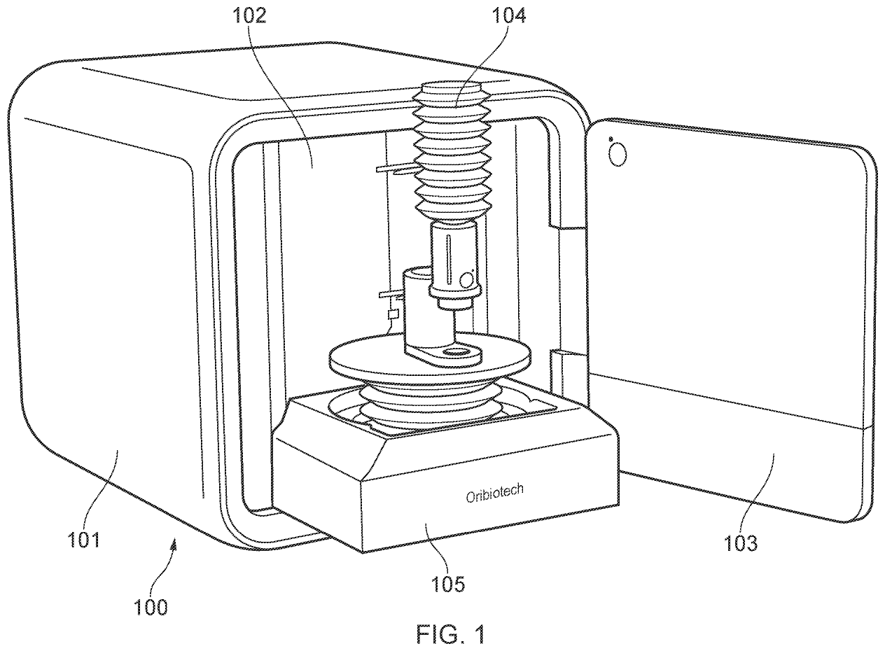

In FIG. 1, a pod 100 is shown. The pod 100 is a discrete incubator unit comprising walls forming a housing 101 with an internal cavity 102 that may be closed by a door 103. The door 103 may be made of a transparent material to allow easy visualization of the pod cavity 102 and its contents, or alternatively, may be made of an opaque or translucent material. A secondary transparent internal door may be provided when using an opaque/translucent external door. This setup may be advantageous if the external door comprises insulation, but quick-viewing of the internal cavity 102 is required. The secondary door allows this quick-visualization but without incurring a rapid drop in temperature inside the cavity. The perimeter of any door 103 may sealingly engage with the walls of the pod 100 to form a substantially fluid-tight fit. Sealing members may be provided on either the door 103 or the pod walls provided that the function of forming a seal on abutting the door 103 against the pod wall is fulfilled. The pod 100 is provided with all necessary actuators to attach, connect, and dispense contents from one or more reservoirs to a bioreactor. The one or more reservoirs may or may not be located within the pod 100. The reservoir(s) may be remote from the pod or an array of pods. The bioreactor is located within the internal cavity 102 of the pod 100. The pod may further comprise means for agitating the contents of the bioreactor (also known as a biological sample vessel 104). Any suitable agitation means may be employed, where that is vibration plates, rocking devices, magnetic stirring (provided the bioreactor is provided with a magnetic stirrer or flea), or any other known means.

The walls of the pod 100 may be of equal dimensions such that the pod 100 is substantially cuboidal in shape, or any alternative dimension necessary may be used in order to accommodate a given biological sample vessel 104. The pods may take any suitable 3D form such as, but not exclusively, those approximating a cube, cuboid, sphere. It will be apparent that any 3D form may be suitable for the pod 100 to take, provided that 3D form allows the pod 100 to be located with other pods to form an array of pods.

The walls/doors of the pod 100 may suitably be insulated in order to maintain an internal temperature suitable for the culture and maintenance of biological samples. Each pod 100 may be provided with its own temperature regulation and heating system, or the array may be provided with a communal temperature regulation and heating system, whereby some or all of the pod cavities in a given array are maintained at a temperature set globally for those pods sharing the communal temperature regulation and heating system.

Pod cavities may be environmentally isolated relative to one another, such that a given pod 100 may have independent maintenance and fluid lines separates from other pods, or those lines pass through filters and the like in order to prevent communication between the pods or prevent cross-contamination. As such the pod cavities may be isolated.

An array may be split into discrete zones. Pods located in those zones may be provided with separate services such as temperature regulation compared to pods in other zones. The zones may be fixed (e.g., limited to a predetermined row and/or column or plural rows/columns), or the zones may be dynamically set in the array on the fly, for example, if a given pod or set of pods required a bespoke heating regime having multiple temperatures for set durations applied during culture of a biological sample the zones may adapt/change as per the culture requirements.

Biological sample vessels 104 are locatable within each pod 100. The pod 100 may comprise a drawer 105 or displaceable shelf (e.g., via sliding) that can be withdrawn from the pod cavity 102 to the external environment. The drawer or displaceable shelf 105 may be actuated manually (i.e., by hand) or mechanically (e.g., motor assisted or motor driven/automated). The use of a shelf/drawer facilitates location of the biological sample vessel 104, via an interfacing element, to a complementary interfacing element located within the pod 100. In this instance, the complementary interfacing element is located upon the drawer or displaceable shelf 105. The interfacing element comprises an interface plate having at least one port to allow the passage of fluids therethrough. A locking means or access control may be provided to prevent inadvertent or unauthorized operation of the drawer/shelf 105 to protect the contents. Any suitable access control or locking means may be used.

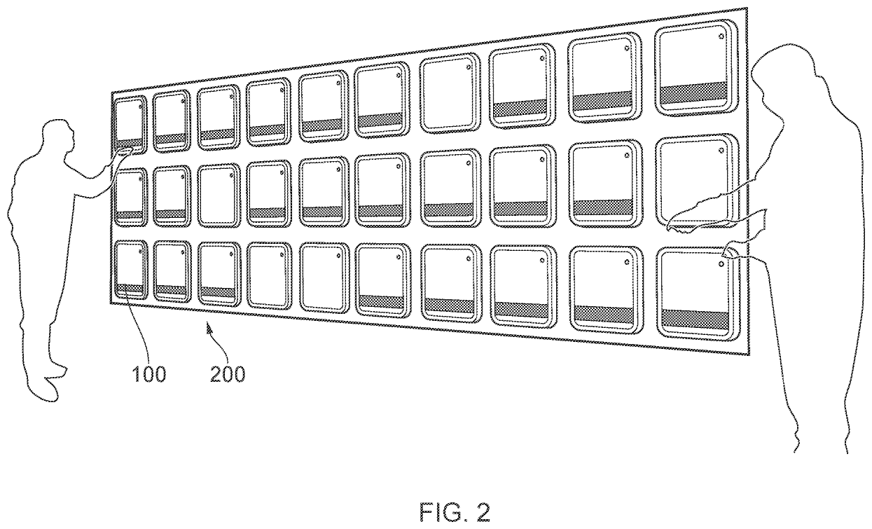

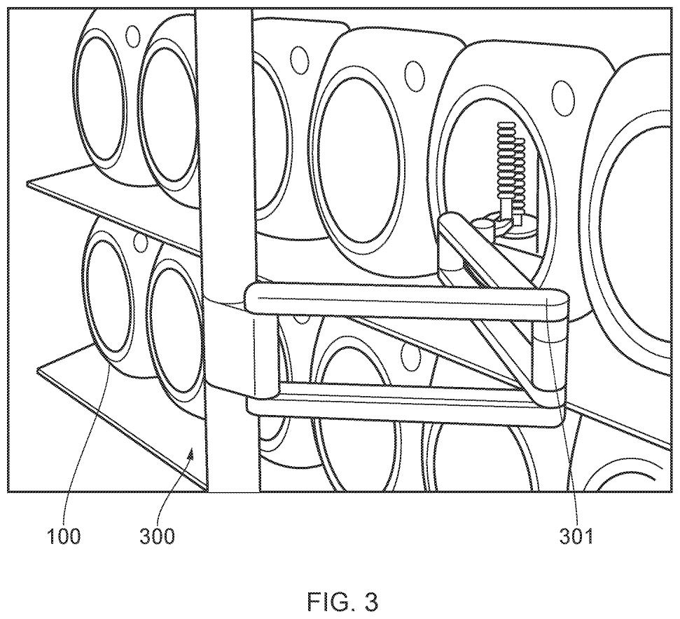

As shown in FIG. 2, a plurality of pods 100 may be arranged into a bank or an array 200 of pods. Each location in the array 200 may have its own unique address in order to facilitate the location and tracking of individual pods 100 and/or the biological samples contained therein. Any suitable addressing system may be used, such as, but not limited to barcodes or radio frequency identification tags. The array 200 comprises rows and columns of pods 100, although other arrangements are acceptable if such arrangements make efficient use of space; for example, the pods 100 may be arranged in concentric circles, in offset rows and/or columns, or a less regular format. The purpose of the array is to maximize space utilization and therefore the shape/form of the array may be in part dictated by the site the pods are located, such as the shape and geometry of the site walls, floor, and ceiling (where any walls, floor, and/or ceiling are present) in which the pods are located. The array 200 may be “fitted” and integrated with the fabric of a facility (i.e., pods are inset into the walls of a building, for example) or “free standing,” with each pod 100 loosely associated with neighboring pods on racking or the like, such as that shown in FIG. 3.

The array 200 shown in FIG. 2 comprises rows of 10 pods and columns of 3 pods; however, it will be apparent that any number of pods 100 may be accommodated in different array 200 patterns to fill an available space in a facility. The facility may be a physical building on a fixed site, or the facility may be a mobile facility such as a unit provided on a trailer bed that may be towed and relocated using a tractor unit such as, but not limited to, an articulated lorry, box truck, Luton van, or the like. It will be apparent that any mobile facility will be appropriate provided the space used to locate the array is suitable to ensure correct and safe biological sample culture and that the tractor unit used to relocate the mobile facility is capable of such safe transportation. It will be apparent that the tractor unit may be integral to, or separable from, the unit of the mobile facility. For the avoidance of doubt, here tractor unit means the means of propulsion capable of moving (towing) a load. Unit means a mobile facility that houses and allows the provision of services to (services may be contained or supplied externally) one or more arrays of pods located within the unit. Units may comprise any of walls, ceiling, floor, and may or may not be fully enclosed depending on whether a successful culture can be achieved with or without these features. Typically, a unit will comprise a floor, walls, and ceiling to form an enclosed space in order to shelter and insulate the array(s) from the outside environment and provide an environmentally controlled and clean space suitable for culture of biological samples within a pod as described above.

FIG. 3 shows an alternative array 300 of pods 100 having a different style. These pods 100 are stand-alone and not fitted with the fabric of the facility allowing easy access for maintenance, installation, and replacement. Such an arrangement also allows the array to be dynamically changed in size with pods 100 added or removed in response to changing manufacturing demands.

FIG. 3 also shows a pod access system 301 that is capable of locating a given pod 100 in an array, driving to that pod 100, and interacting with the pod 100 and the contents of the pod's cavity. Interactions could include decoupling part or all of the contents of the pod 100 (such as the biological sample vessel 104 or any reservoirs and consumables located within the pod 100), from the array, gripping all or a part of the pod contents, and transporting the contents to a remote location.

It will be apparent that the complementary interfacing element may be placed at any convenient location within the cavity 102 of the pod 100; preferably, the complementary interfacing element is placed in a location to facilitate coupling with the interfacing element of the biological sample vessel 104. As can be seen by way of example, in FIG. 1, the complementary interfacing element is disposed toward a base of the pod cavity 102 with the interfacing element of the biological sample vessel 104 located toward the bottom of the vessel. It will be apparent that it is not essential that this is the case and any suitable arrangement may be used instead.

The interfacing element provides communication to the interior of the pod 100 and/or the biological sample vessel 104, providing access to fluids required for the maintenance of the biological sample under culture, such as, but not exclusively, an auxiliary vessel, a gas regulatory system, a waste management system, a sterilization system, and a reservoir containing culture fluid. Auxiliary vessels may be used to decant an expanding culture, for example, which can then be transported to a separate pod 100 for further expansion of the biological sample. The gas regulation system would provide a fresh and constant supply of fresh gas in order to facilitate pH regulation with culture media.

The biological sample vessel 104 may be a flexible container made of a polymer. The vessel could take the form of a flexible bag or pouch, or as shown in FIGS. 1 and 3, be a plastic bottle with corrugated walls such that the vessel folds up concertina-style. As such the vessel can accommodate varying volumes of fluids and be readily emptied or filled without a substantial change in internal pressure within the limits of capacity of the vessel (the limits being from a fully expanded volume to a volume of the vessel when fully contracted). Preferably, the biological sample is contained within the vessel to maintain sterile conditions as well as to allow for easy handling and transportation, as the vessel may be removably attached to the pod 100 via the interface element.

In order to culture a biological sample using the disclosed system, one or more biological samples are first loaded into one or more pods. The sample may suitably be provided in a biological sample vessel 104. Once the sample is located in the pod 100, the biological sample is then cultured. When in the pod 100, the interface element of the biological sample vessel 104 is connected to the complementary interfacing element of the pod 100 in order to allow fluid exchange in order to maintain the biological sample.

Throughout the description and claims of this specification, the words “comprise” and “contain” and variations of them mean “including but not limited to,” and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the disclosure are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents that are filed concurrently with or previous to this specification in connection with this disclosure and that are open to public inspection with this specification, and the contents of all such papers and documents are incorporated herein by reference.

1. A bioreactor system, comprising:
a plurality of pods, the pods suitable for the culture and maintenance of biological samples, each pod comprising:
a housing, and
a biological sample vessel removably disposed within the housing, the biological sample vessel comprising an interfacing element to provide fluid communication to the biological sample vessel,
wherein the plurality of pods are arranged in an array.

2. The bioreactor system as claimed in claim 1, wherein the interfacing element provides fluid communication to one or more of: an auxiliary vessel, a gas regulatory system, a waste management system, a sterilization system, and a reservoir containing culture fluid.

3. The bioreactor system as claimed in claim 1, wherein the biological sample vessel comprises a flexible container.

4. The bioreactor system as claimed in claim 3, wherein the flexible container comprises a biological sample.

5. The bioreactor system as claimed in claim 3, wherein the flexible container is configured to removably attach to a pod of the plurality of pods.

6. The bioreactor system as claimed in claim 1, wherein the interfacing element comprises an interface plate, coupled to the biological sample vessel, the interface plate including at least one port.

7. The bioreactor system as claimed in claim 1, wherein each of the plurality of pods is positioned in the array to be uniquely addressable.

8. The bioreactor system as claimed in claim 1, wherein each of the plurality of pods is removably located within the array.

9. The bioreactor system as claimed in claim 1, wherein an interior of each of the plurality of pods is isolated from the interior of other pods of the plurality of pods in the array.

10. The bioreactor system as claimed in claim 1, wherein each of the plurality of pods are removably attached within the array.

11. The bioreactor system as claimed in claim 1, wherein the array is formed integrally with a building.

12. The bioreactor system as claimed in claim 1, wherein bioreactor system is provided in a mobile facility.

13. The bioreactor system as claimed claim 1, wherein the plurality of pods arranged in the array form at least one of rows and columns.

14. The bioreactor system as claimed in claim 1, wherein the array is chosen from among: a 2D array; a 3D array; a horse-shoe array; a spherical array; and an irregular array.

15. A method of culturing a biological sample, comprising:
loading at least one biological sample into at least one pod of a bioreactor system, the bioreactor system comprising:
a plurality of pods, the pods suitable for the culture and maintenance of biological samples, each pod comprising:
a housing, and
a biological sample vessel removably disposed within the housing, the biological sample vessel comprising an interfacing element to provide fluid communication to the biological sample vessel,
wherein the plurality of pods are arranged in an array;
arranging the at least one pod in the array; and
culturing the at least one biological sample.

16. The method of claim 15, further comprising one or more of:
loading at least one consumable product into one or more pods of the plurality of pods;
removing the at least one consumable product from the one or more pods;
retrieving the at least one consumable product from storage;
removing the at least one consumable product to waste;
loading at least one auxiliary service into a pod of the plurality of pods;
retrieving a test sample from the biological sample vessel; and
loading one of a sensor or test apparatus to perform an observation on a condition inside the pod of the plurality of pods.

17. The method of claim 16, wherein the consumable product is chosen from among a container for fluid dispensation and a container for sample collection.

18. A kit of parts to form a bioreactor system, the kit of parts comprising:
a plurality of pods, the pods suitable for the culture and maintenance of biological samples, each pod comprising:
a housing, and
a biological sample vessel removably disposed within the housing, the biological sample vessel comprising an interfacing element to provide fluid communication to the biological sample vessel,
wherein the plurality of pods are arranged in an array.

19. The bioreactor system as claimed in claim 10, wherein each of the plurality of pods is removably attached from one of a first side of the array and a second side of the array, the first side of the array and the second side of the array opposite one another.

20. The bioreactor system as claimed in claim 3, wherein the flexible container comprises a container chosen from among a flexible bag, a pouch, and a bottle including corrugated walls.
